(19) United States
(12) Patent Application Publication
(10) Pub. No.: US 2013/0023768 A1
(43) Pub. Date: Jan. 24, 2013

(54) ULTRASOUND PROBE POSITIONING IMMERSION SHELL

(71) Applicants: RAMIN HOORIANI, CALABASAS, CA (US); RAFI ISRAEL, CALABASAS, CA (US)

(72) Inventors: RAMIN HOORIANI, CALABASAS, CA (US); RAFI ISRAEL, CALABASAS, CA (US)

(21) Appl. No.: 13/185,790

(22) Filed: Jul. 19, 2011

(51) Int. Cl. A61B 8/10

(52) U.S. Cl. 600/459

(57) ABSTRACT

Proposed is an improved ultrasonic immersion shell made of soft biocompatible material which forms a water-tight seal when attached to an ultrasonic A-Scan probe. When properly filled with fluid, it utilizes a convex liquid dome to form a meniscus between itself and the cornea in order to eliminate any corneal compression during immersion biometry and maintaining majority of the fluid within its immersion chamber once removed from the eye therefore eliminating the need for any cleanup of excess fluid.

### FIELD OF INVENTION

This invention relates generally to the ultrasonic measurement of axial length, anterior chamber depth and retinal detail of the eye and is particularly directed to the measurement of the structures in an eye through a water bath, also known as immersion A-Scan biometry.

### PRIOR ART

There are several situations in the treatment of an ophthalmic patient that require a diagnostic ultrasound examination providing detailed information of the anatomical structures of the eye. This information enables the physician to provide the best possible care for a large variety of ocular disorders.

The most frequent use of ultrasound in ophthalmology is the axial eye length A-scan used to measure the eye prior to cataract surgery. A synonym for this type of A-scan is biometry. This measurement of an eye's axial length provides one of the three important values needed to calculate the appropriate power of an intraocular lens (IOL) implant after cataract removal. An inaccurate axial length measurement of just one millimeter can result in a post-operative optical error of three diopters, enough to necessitate a second surgery. Cataract removal and insertion of an intraocular lens (IOL) is performed over 1.5 million times a year in the U.S.

Two of the most commonly used techniques to perform axial eye length measurements are as follows:
a. Applanation, a contact technique; and,
b. Immersion, an ultrasound through a water bath technique.

With the contact method, the axial length is measured with the ultrasound probe applanated on the center of the cornea. The biometrist must ensure that neither ointment nor excess fluid (e.g., anesthetic drops or tears) are present on the cornea prior to beginning the examination, since even a small amount of fluid may lead to erroneous axial length readings. The contact technique can be performed by applanation (chin rest method) or by hand (hand-held method).

Disadvantages of the contact technique include both corneal compression and the possibility of corneal abrasion. The anterior chamber depth must be evaluated in each echogram since shallowing of the chamber occurs when the cornea is indented. Further, due to examiner parallax or alignment problems, it is often difficult to be sure measurements are taken from the center of the cornea, producing a falsely short measurement. This technique is also best for patients with blepharospasm and fixation difficulties.

The three fundamental advantages of the immersion method are the following:
a. the capability to prevent inaccuracies due to corneal compression by eliminating the need to touch the cornea;
b. the capability to reproduce the measurement more readily; and,
c. the capability to use echoes from the cornea for aligning the sound beam along the visual axis, thereby providing additional assurance of a measurement to the macula.

With the advancement in IOL design and manufacturing, a more precise axial length measurement of the eye is required for determining the correct IOL power required for optimal pseudophakic correction. An inaccurate axial length measurement of only 1 mm can result in a significant post-operative refractive error. The ultrasound manufacturers have improved the A-scan equipment with upgraded hardware and software for measuring eye length with the transducer immersed in a liquid medium. The biometry instrument converts the time readings into millimeter axial length. When using the immersion technique, these A-scan improvements require that the ultrasound probe tip be placed at a fixed and specified distance from the corneal surface. For accurate measurements, it is essential that the ultrasound probe remains perpendicular to the visual axis while the transducer is submersed. It is equally important for the liquid medium between the corneal surface and the transducer to be free of trapped air bubbles. The presence of air bubbles can disrupt the sound wave transmission and interfere with axial length measurement.

To keep the eye submersed in a liquid medium during biometry, various cylindrical shells of different shapes are used in immersion A-scan. All have shortcomings.

### Hansen Shell

The Hansen shell is simply a plastic cylinder open at both ends incorporated in a two-handed procedure requiring skill to master. The Hansen shell is inserted under the eyelids and hand-held while the liquid medium is poured from the top submerging the transducer and eye. Because the ultrasound probe is free to move, it can be easily moved vertically and tilted, resulting in erroneous measurements. Further, a viscous solution, Goniosol, is required, which is expensive and leaves a vision-blurring film. Achieving accurate measurements using this shell design is difficult to master.

### Kohn Shell

The Kohn shell has an hourglass shape with the ultrasound probe inserted to the constriction. A port including a metal tube and hose is located at the bottom portion or lower chamber of the shell for introducing the liquid medium. The ultrasound probe and shell meet at one location with a larger diameter opening at the top of the shell. This can result in vertical and angled error due to a single fulcrum contact point, as with mating two cones with different dimensions and angles. Any dimensional difference between the ultrasound probe shape and the shell constriction increases the likelihood for the ultrasound probe to be tilted and/or positioned at a different height.

This Kohn shell design forms two chambers once the ultrasound probe is inserted, and a “cork effect” occurs at the contact point between the ultrasound probe and shell constriction. The liquid medium then must be injected after the shell is on the eye through the port located in the lower chamber between the constriction and the bottom of the shell contacting the eye. This reduces the ability to visually place the shell and ultrasound probe on the eye due to the port and any connected tubing blocking the view. Furthermore, due to the ultrasound probe blocking the air from escaping from the lower chamber, a large air bubble can be easily trapped in the lower chamber and prevent an ultrasound axial length measurement.

### Prager Scleral Shell

Another current design is the Prager scleral shell. This is a polycarbonate plastic cylinder with a flanged end that contacts the eye. To accommodate the ultrasound probe, the upper portion of the shell is bored out in the center with an inner diameter slightly larger than the ultrasound probe to maintain orientation. A setscrew located at the top of the shell is then tightened against the probe to preclude the probe from protruding out the shell bottom and potentially contacting the cornea. The probe tip can be placed at any height from the cornea. This requires the operator to carefully inspect the ultrasound probe height position before every ultrasound exam. If the ultrasound probe tip is either too low or too high, a faulty reading will occur. Furthermore, the ultrasound probe can be easily canted from the perpendicular position in the shell when the setscrew is tightened. To fill the shell with the liquid medium, a metal port or filler tube is press fit into the shell wall. The metal port or filler tube is inserted into PVC tubing or a butterfly needle is inserted into the metal port or filler tube. Any sharp object in close proximity to the eye is considered a safety issue.

Typically, multiple holes are drilled into the wall of the lower portion of the shell for air to escape as the liquid enters the lower portion of the shell.

Draw backs to current immersion shells in the market today include:
a. It is difficult technique to perform for the user and placement of the shell on the sclera is uncomfortable for the patient. The operator has to manage to hold the shell on the sclera forming a water tight seal, and then fill the shell with the required amount of liquid while holding the ultrasonic probe in the center of the shell at the right distance form the cornea and along the visual axis of the cornea. It is difficult to train people on this technique and there is a strong resistance in the eye care market to its adoption.
b. Since different people have different size eyes, one shell does not fit all eyes and immersion biometry becomes an issue especially for kids who have smaller eyes.
c. There is no mechanism for removing the coupling liquid from the immersion chamber after the completion of the test. This results in the spillage of liquid on the patient's face when the test has been completed and the shell is removed.
d. The same shell is used on multiple patients which increases the possibility of patient cross contamination.
e. To disinfect the sclera shell it must be autoclaved or submersed in alcohol for several minutes which can lower an office's efficiency.

### BRIEF SUMMARY AND OBJECTIVES OF INVENTION

The general purpose of the present invention is to provide an ultrasound probe positioning immersion shell. To achieve the objectives for correct ultrasound probe position during immersion AScan, a unique immersion shell has been developed, the ultrasound probe positioning immersion shell consistently places the ultrasound probe perpendicular to and at the correct distance from the corneal plane. The ultrasound probe positioning immersion shell uses the water tight seal between itself and the ultrasound probe to make a water tight chamber. The ultrasound probe positioning immersion shell is design to be filled directly with fluid to minimize air bubbles, and to hold the fluid with a small convex fluid dome meniscus using the principle of water surface tension. The ultrasound probe positioning immersion shell shall be filled with fluid prior to patient contact, the diameter of the ultrasound probe positioning immersion shell shall be such that it maximizes the convexity of the fluid when the chamber is filled, the tip of the chamber is design to minimize the transfer of fluid to the patient. Additionally there is a reservoir chamber that prevents the leakage of the fluid and provides a reservoir of fluid for contact with the cornea.

One significant aspect and feature of the present invention is an ultrasound probe positioning immersion shell that is fully water sealed when properly placed on the A-Scan transducer probe. This seal insures perpendicularity of AScan probe to the plane of the immersion shell tip.

Another aspect of the present invention is that it is made of soft compressible medical grade material that minimizes the patient discomfort, risk of corneal abrasion, and indentation of the eye.

Another aspect of the present invention is that it is single use, sanitized and or sterile, and disposable, therefore minimizing the patient cross contamination.

Another aspect of the present invention is that it uses the principle of the water surface tension to hold a convex dome of water meniscus.

Yet another significant aspect and feature of the present invention is that the chamber is filled prior to contact to the patient eye.

Yet another significant aspect and feature of the present invention is geometry which limits the travel of an ultrasound probe along the central axis and acts as a stopper.

Yet another aspect and feature of the present invention is in an ultrasonic positioning immersion shell which positions the probe at a precise distance form the tip of the shell.

Another aspect of the present invention is that the dome of fluid is used as the material to contact the cornea hence alleviating the corneal compression.

Another significant aspect and feature of the present invention is that the software in the AScan device will constantly monitor the distance between the AScan probe and the cornea and warn users if the corneal echo has crossed an minimum distance threshold.

Yet another feature of the present invention is that it prevents the ultrasound probe from invading (contacting) the cornea of the eye.

Yet another significant aspect and feature of the present invention is the design of the inward lip at the tip of the immersion chamber to reduce the transfer of the fluid to the patient.

Yet another significant aspect and feature of the present invention is the design of the inward lip at the tip of the immersion chamber to maximize the stability of liquid meniscus dome.

Yet another significant aspect and feature of the present invention is its inward lip design of the immersion chamber that minimizes formation of air bubbles.

Yet another significant aspect and feature of the present invention is its transparent design to enable users to see any air bubbles.

Yet another significant aspect and feature of the present invention is the size and geometry of the immersion chamber, designed to be able to form the convex water meniscus dome using the water surface tension principle.

These and other objects and features of the present invention will be apparent from the following detailed description, taken with reference to the figures of the accompanying drawing.

### BRIEF DESCRIPTION OF THE DRAWINGS

FIG. 1 is an isometric view of an immersion shell embodying the present invention, shown removed from the ultrasonic A-Scan probe;

FIG. 2 is a cross sectional view of the immersion shell of FIG. 1 depicting the internal structures of the present invention;

FIG. 3 is a picture of the immersion shell embodying the present invention, shown attached to an ultrasonic A-Scan probe with its immersion chamber filled with liquid.

FIG. 4 is a picture of the immersion shell embodying the present invention, shown attached to an ultrasonic A-Scan probe with its immersion chamber filled with liquid as it is approaching a human cornea for the purpose of measuring the structures within the eye.

### DETAILED DESCRIPTION OF THE INVENTION

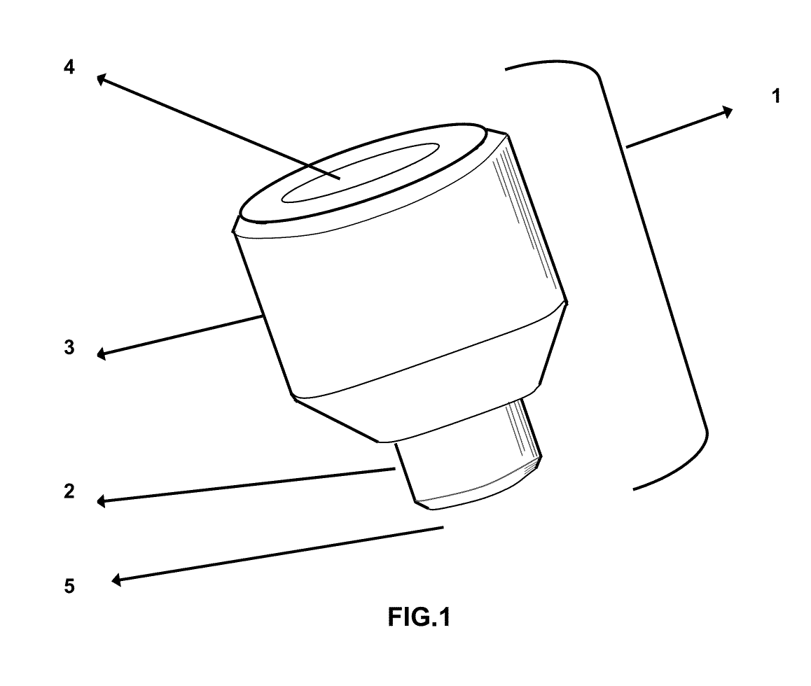

In that form of the present invention chosen for purposes of illustration, FIG. 1 shows an immersion shell indicated generally at 1, comprising an immersion chamber 2, an ultrasonic probe chamber 3, a probe opening 4 for receiving an ultrasonic probe, and an immersion chamber opening 5 for receiving liquid in order to fill the immersion chamber 2 and interfacing the device to a patient's eye.

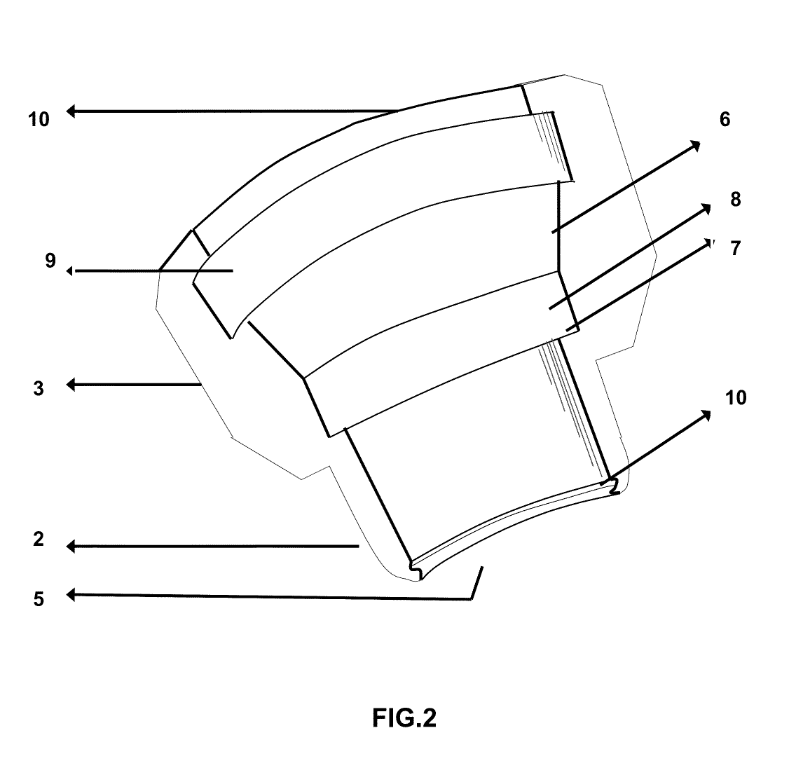

In that form of the present invention chosen for purposes of illustration, FIG. 2 shows a cross sectional view of the immersion shell 1 comprising a Probe Guide 6 with function of centering the probe as it is inserted in to the immersion shell 1, a Probe Stop 7 that limits the distance the probe is inserted into the immersion shell 1, a probe seal 8 that creates an water tight seal around the probe, an excess liquid compartment 9 that traps any additional liquid that has escaped the immersion chamber 2, and an Immersion Chamber Inward Lip 10 that helps stabilize the immersion chamber 2 liquid dome.

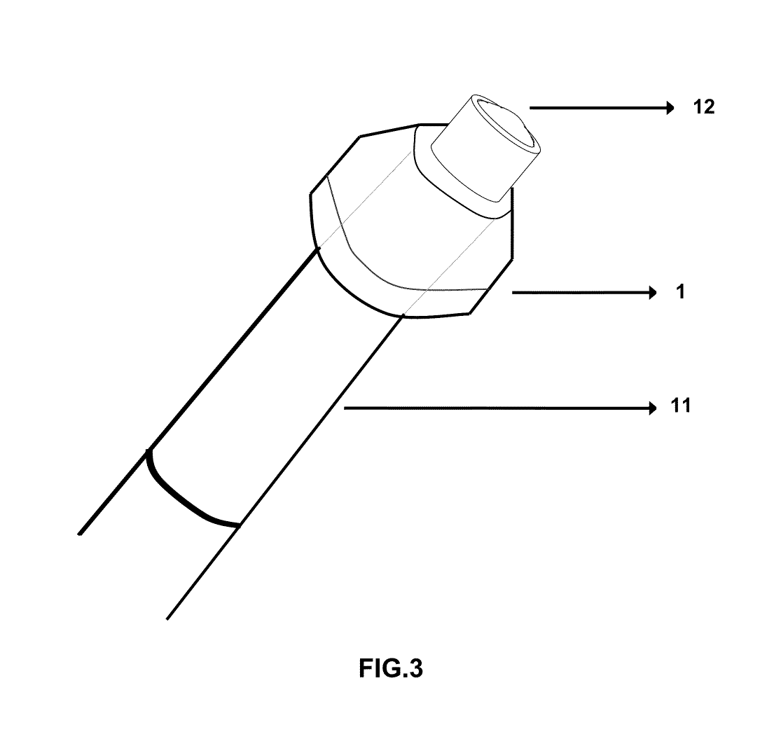

FIG. 3 depicts the proposed immersion shell 1 attached to an A-Scan Probe 11 with the immersion chamber 2 filled with a liquid to create a convex liquid dome 12.

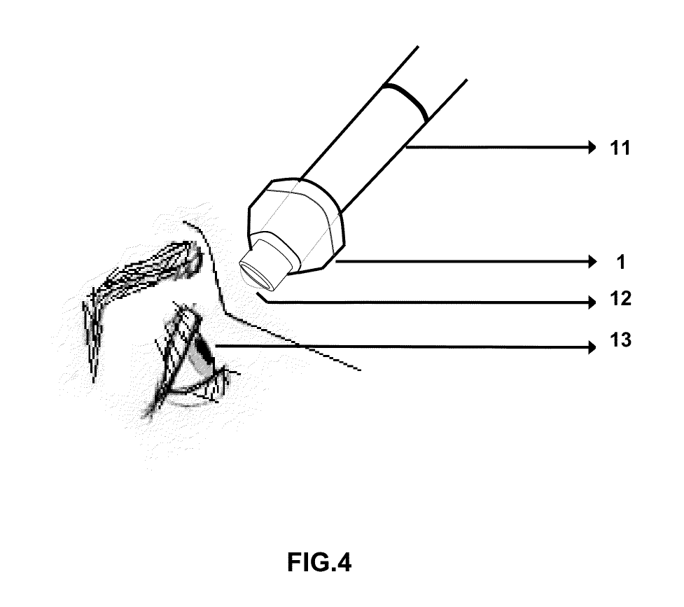

FIG. 4 depicts the proposed immersion shell 1 attached to an A-Scan Probe 11 with the immersion chamber 2 filled with a liquid to create a convex liquid dome 12 as it approaches a human cornea 13 for the purpose of performing immersion biometry.

The proposed immersion shell of FIG. 4, in one embodiment can be made of a very soft medical grade material in order to minimize the possibility of corneal compression and aberration. In use, the immersion shell 1 will be placed on an A-Scan probe 11, with the immersion shell 1 parallel in axis with the A-Scan probe 11. As best seen in FIG. 2, the ultrasonic probe 11 is inserted through the Probe Opening 4, through the Probe Guide 6, and Probe Seal 8, up to the Probe Stop 7 forming a water tight seal between the immersion chamber 2 and the ultrasonic probe 11. As shown in FIG. 3, this water tight seal along with the size and geometry of the proposed immersion shell, takes advantage of the surface tension principle of the liquid in the immersion chamber 2 to form a convex Dome 12 of liquid at the opening of the immersion chamber 5.

Once the proposed immersion shell 1 is properly engaged to a A-Scan probe 11 and is filled with liquid and a convex dome 12 is created, the immersion shell 1 can retain this liquid dome 12 regardless of the direction in which the probe is held.

In use, as seen in FIG. 4, the immersion shell 1 attached to an A-Scan probe 11 and filled with proper amount of liquid to form a convex dome 12 is brought to contact with the cornea 13. This convex dome 12 of liquid forms a meniscus of fluid separating the immersion shell 1 from the cornea 13 and eliminating any corneal compression. When the measurement of the eye is complete, the user retracts the AScan probe 11, immersion shell 1 assembly from the eye. The inward lip 10 of the immersion shell 1 helps maintain majority of the fluid in the immersion chamber 2 of the proposed shell.

The proposed geometrical design of the immersion chamber 2 minimizes the amount of fluid used for each measurement and this design is capable of maintaining the fluid in its immersion chamber 2 during use. As a result, in most instances a minimum amount of fluid is transferred to the eye. This eliminates the need for clean up of excess fluid upon the completion of biometry.

Obviously, numerous variations and modifications can be made without departing from the spirit of the present invention. Therefore, it should be clearly understood that the forms of the present invention described above and shown in the figures of the accompanying drawings are illustrative only and are not intended to limit the scope of the present invention.

What is claimed is:

1. An ultrasound probe positioning immersion shell 1 comprising an immersion chamber 2, an ultrasonic probe chamber 3, a probe opening 4 for receiving an ultrasonic A-Scan probe 11, and an immersion chamber opening 5, a probe seal 8 characterized in that said immersion shell 1 is attached to said ultrasonic A-Scan Probe 11 with said immersion chamber opening 5 receiving liquid and filling to create a convex liquid dome 12 that interlaces said immersion shell 1 to a human eye cornea 13 during immersion biometry.

2. An immersion shell as claimed in claim 1 wherein said ultrasonic A-Scan probe 11 is inserted through said Probe Opening 4 and said Probe Seal 8, up to a Probe Stop 7 forming a water tight seal between said immersion chamber 2 and said ultrasonic A-Scan probe 11.

3. An immersion shell as claimed in claim 1 wherein a Probe Guide 6 centers said ultrasonic A-Scan probe 11 as it is inserted in to the immersion shell 1.

4. An immersion shell as claimed in claim 1 wherein said Probe Stop 7 limits the distance said ultrasonic A-Scan probe 11 is inserted into said immersion shell 1.

5. An immersion shell as claimed in claim 1 wherein said immersion chamber opening 5 receives liquid prior to patient contact and forms said convex liquid dome 12 at the opening of said immersion chamber 5 through surface tension principle, the diameter of said immersion shell 1 maximizing the convexity said convex liquid dome 12 when said immersion chamber 2 is filled.

6. An immersion shell as claimed in claim 1 wherein said probe seal 8 creates a water tight seal around said ultrasonic A-Scan probe 11.

7. An immersion shell as claimed in claim 1 wherein an excess liquid compartment 9 traps any additional liquid that has escaped said immersion chamber 2.

8. An immersion shell as claimed in claim 1 wherein an Immersion Chamber Inward Lip 10 stabilizes said convex liquid dome 12 at the opening of said immersion chamber 5 and retain said convex liquid dome 12 regardless of the direction in which said ultrasonic A-Scan probe 11 is held.

9. An immersion shell as claimed in claim 1 wherein said immersion shell 1 positions said ultrasonic A-Scan probe 11 at a precise distance from the tip of said immersion shell 1.

10. An immersion shell as claimed in claim 1 wherein a customized software constantly monitors the distance between said ultrasonic A-Scan probe 11 and said human eye cornea 13 and warns users if the corneal echo has crossed an minimum distance threshold.

11. An immersion shell as claimed in claim 1 wherein said ultrasound A-scan probe is placed perpendicular to and at the correct distance from the corneal plane during immersion biometry, said convex liquid dome 12 forming a meniscus of fluid separating said immersion shell 1 from said human eye cornea 13.

12. An immersion shell as claimed in claim 1 wherein said immersion shell 1 is made of soft medical grade biocompatible silicone material that forms a water-tight seal when attached to said ultrasonic A-Scan probe 11.

13. An immersion shell as claimed in claim 1 wherein said immersion chamber 2 structure, minimizes the amount of fluid used for each measurement and maintains the fluid in its immersion chamber 2 during use.

14. An immersion shell as claimed in claim 1 wherein said Immersion Chamber Inward Lip 10 structure maximizes the stability of and minimizes the transfer of fluid from said convex liquid dome 12 to said human eye cornea.

15. An immersion shell as claimed in claim 1 wherein said Immersion Chamber Inward Lip 10 minimizes formation of air bubbles in said Immersion Chamber 2.

16. An immersion shell as claimed in claim 1 wherein said immersion shell 1 is made of transparent material that shows air bubbles formed during filling of said immersion shell 1.
